(19) United States
(12) Patent Application Publication
(10) Pub. No.: US 2008/0287854 A1
(43) Pub. Date: Nov. 20, 2008

(54) Emergency-Disengagement Device for Patients Undergoing Hemodialysis

(76) Inventor: Jiandong Sun, San Jose, CA (US)

Correspondence Address: Jiandong Sun, 1649 Merrill Drive #3, San Jose, CA, 95124, US

(21) Appl. No.: 11/426,307

(22) Filed: Jun. 24, 2006

(51) Int. Cl. A61M 1/14

(52) U.S. Cl. 604/6.15

(57) ABSTRACT

An Emergency-Disengagement Device (EDD) comprises three holders (11), into which two blood tubes (21) are inserted. When immediate evaluation is required, patients undergoing hemodialysis can turn the trigger (20) to release sliding blocks (15) (16) in the three holders to choke off and sever the two blood tubes (21), at which time the EDD disassembles to three individual pieces. After the EDD disassembles, patients can rapidly and safely disengage from the hemodialysis machines to which they are connected, without any bleeding.

The invention named Emergency-Disengagement Device (EDD) is an attachment to kidney hemodialysis equipment. When an emergency occurs that requires immediate evacuation, the EDD enables patients undergoing hemodialysis to rapidly and safely disengage from the hemodialysis machines to which they are connected without assistance from other people.

Neither the idea nor a similar object to the EDD has ever been found in renal clinics or in any relevant literature.

### BACKGROUND OF THE INVENTION

1. Technical Field

The present invention relates to choking off and severing the two blood tubes connecting a patient to a hemodialysis machine without causing bleeding, and thus allowing the patient undergoing hemodialysis treatment to rapidly and safely disengage from the hemodialysis machine to which his blood vessels are connected.

2. Description of the Relevant Art

To start hemodialysis treatment, a patient is seated. Two hemodialysis fistula needles are inserted into the patient's fistula or graft within his body. Then a pair of hemodialysis blood tubes connects the two fistula needles to a hemodialysis machine, rendering him immobile. In the case of an emergency, the patient would find it difficult to rapidly disengage from the machine and thus may not be able to escape from a dangerous situation in a timely manner. Careless disengagement of a patient from the hemodialysis machine can lead to massive blood loss and even death of the patient.

Currently, dialysis clinics succor patients in emergency situations (such as fires, earthquakes or terrorist attacks) in the following steps:

1. In order to separate a patient from a hemodialysis machine to which he is connected, a medical staff member or the patient himself must first find an emergency kit, which usually hangs on the hemodialysis machine or the chair in which the patient is seated.

2. Open the emergency kit to retrieve four clamps and a pair of scissors.

3. Use the four clamps to choke off the two hemodialysis blood tubes, each of which needs two clamps to choke off the blood flow.

4. Use the scissors to cut off the hemodialysis blood tubes between the clamps to disengage the patient from the hemodialysis machine.

5. After every patient disengages from the hemodialysis machine and evacuates, the medical staff can evacuate the danger zone.

This procedure calls for skill and time. Even with proper access to emergency kits, many patients would not be able to correctly use the clamps and scissors in an emergency situation. Furthermore, patients during the hemodialysis treatment will only have one arm available to perform the disengagement procedure, making it very challenging to complete. As a result, the medical staff has to help the patients one by one to complete the above procedure, and the evacuation of patients and medical staff would be seriously delayed.

On the other hand, the EDD allows patients to disengage themselves from hemodialysis machines rapidly and easily without any bleeding and without the help of medical staff in an emergency situation.

The following figures give more detailed description of the device.

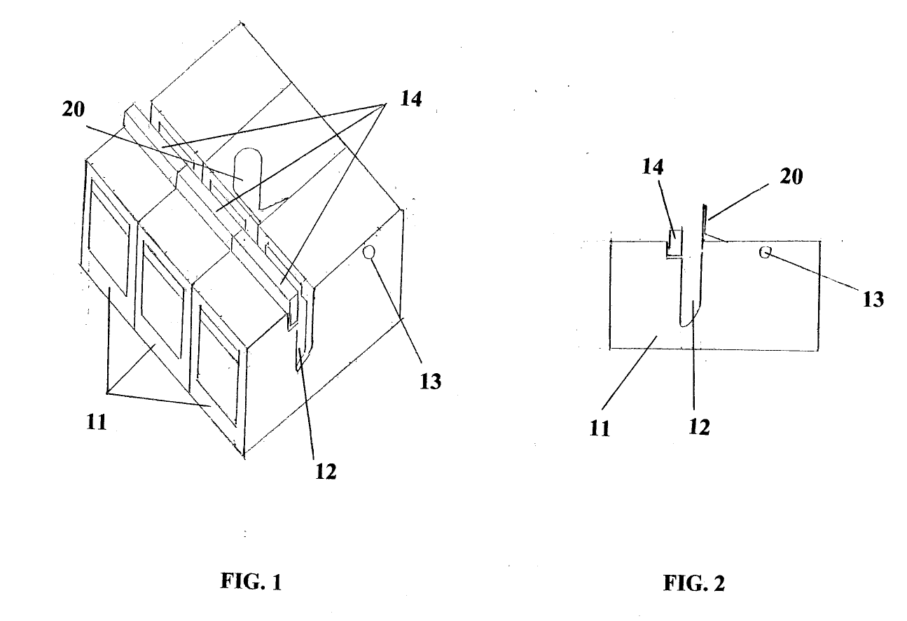

FIG. 1 shows the EDD in a ready-to-work state

FIG. 2 shows a side view of the EDD

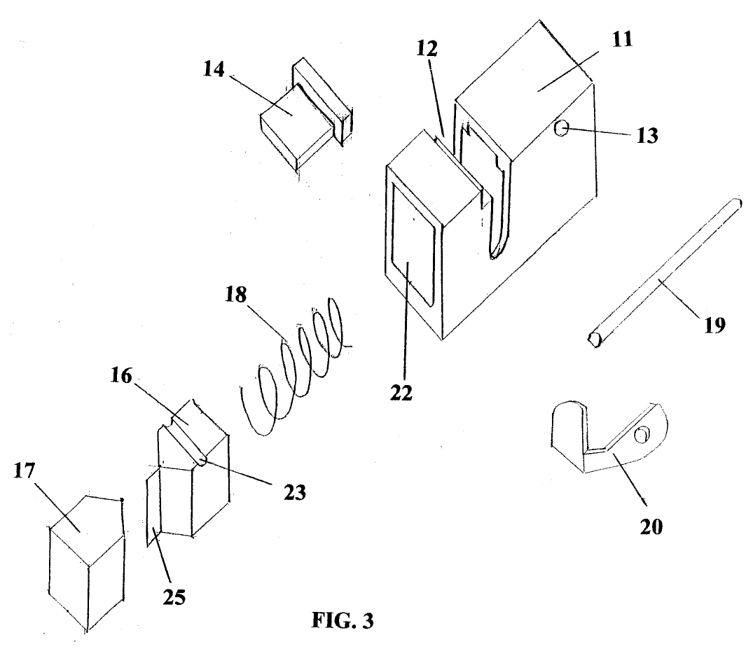

FIG. 3 shows the exploded view of the EDD

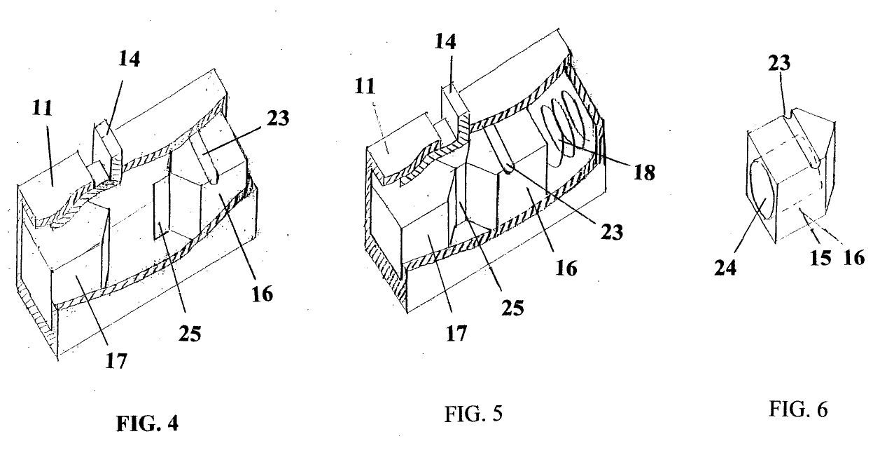

FIG. 4 shows the sectional view of the middle holder while the sliding block compresses the spring against the blind end of the cavity

FIG. 5 shows the sectional view of the middle holder while the sliding cutter moves down and severs the blood tubes

FIG. 6 shows a sliding block

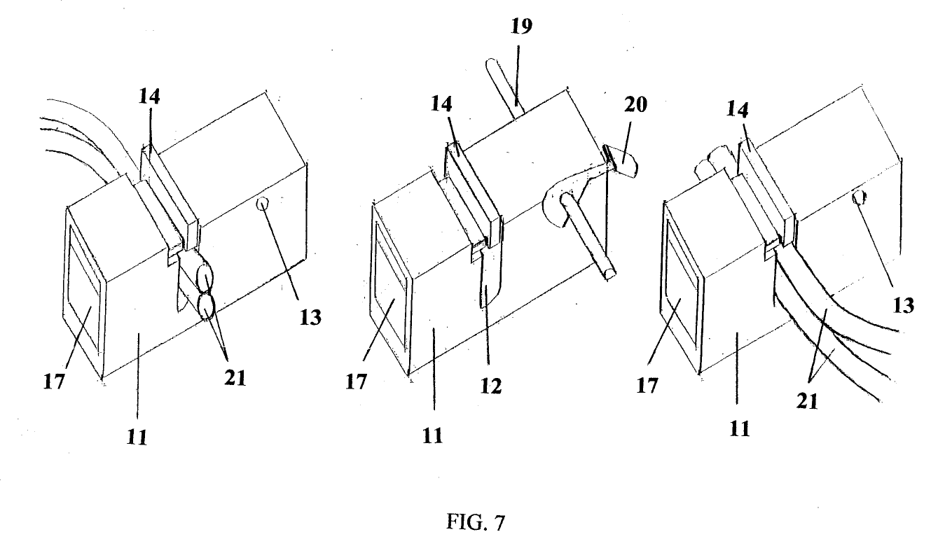

FIG. 7 shows the EDD disassembled after choking off and severing the blood tubes

### DRAWING-REFERENCE NUMERALS

11. Single holder
12. The groove of the holder, into which the blood tubes are placed
13. Cam shift hole
14. Restraint lid
15. Sliding block—sliding choker
16. Sliding block with cutting blade—sliding cutter
17. Fixed block
18. Spring
19. Special-shaped cam shift
20. Trigger
21. Blood tube
22. Open end of the cavity of holder
23. Notch
24. Cavity of sliding block
25. Cutting blade

The EDD consists of three tube holders, three springs, three sliding blocks and three fixed blocks. Each holder has a groove, a square cavity and a cam shift hole. The groove on the holders allows two blood tubes to be placed. The square cavity of the holder is the sliding channel of the sliding block and also the seat of the fixed block. The three holders are held flush against one another by a special-shaped cam shift through the shift hole.

Each sliding block has a notch on the top side that matches to the cam shift and the cam shift holes on the holders. Both the sliding block and the fixed block are ridged on one side and flat on the other side. A spring is located in a cavity on the flat side of the sliding block. A blade is embedded in one of the sliding blocks along the ridge line to form a sliding cutter, while the other two sliding blocks without blades function as sliding chokers. The sliding cutter is placed in the cavity of the middle holder and the sliding chokers are placed in the cavities of the side holders, respectively, each of which compresses a spring against the blind end of the cavity, leaving the groove free. Once the cam shift is inserted into the shift holes on the holders passing through the notches on the sliding blocks, the sliding blocks are locked in place and the three holders are held flush against one another by the pressure of the springs on the cam shift. The sliding cutter is hidden in the holder, so it can not hurt a person or damage the blood tubes.

A trigger handle is installed onto the cam shift between the middle holder and one of side holders. The fixed blocks are placed at the open end of the cavity of the holders, flush with the side surface of the groove and locked. The two blood tubes can be inserted into the groove one over another. The cam shift also controls, through the trigger handle, the release of the sliding chokers and the sliding cutter simultaneously to choke off and sever the blood tubes.

When a patient is ready to start his hemodialysis treatment, a medical staff member or the patient himself installs an EDD by loading the two blood tubes into the groove between the sliding blocks and the fixed blocks and then closing the restraint lids to keep the tubes in place.

To disengage a patient from the hemodialysis machine in an emergency, the patient himself can turn the trigger handle of the cam shift to the end position so that the cam shift releases all three sliding blocks simultaneously. The sliding chokers choke off the two blood tubes against the corresponding fixed blocks in both side holders, while the sliding cutter in the middle holder moves toward its corresponding fixed block and severs the blood tubes. Once the sliding blocks are released, the pressure of the springs on the cam shift is released. The three holders are no longer held together by the cam shift.

The three holders disassemble without causing bleeding in the patient. One of the holders remains attached to the patient, allowing him to disengage from the hemodialysis machine and evacuate the danger zone.

### Instructions:

1. When a patient is ready to start his hemodialysis treatment, a medical staff member or the patient himself installs an EDD by loading the two blood tubes into the groove of the EDD and then closing the restraint lids to keep the tubes in place.

2. In normal situations without any emergency, after hemodialysis is finished a medical staff member or the patient himself can easily open the restraint lids and release the blood tubes from the groove of the EDD. The EDD can be reused.

3. When an emergency occurs (such as fire, earthquake, or terrorist attack), the patient himself can hold the EDD with his available hand and turn the trigger handle to release the sliding blocks, choking off the tubes at both ends of the EDD and cutting the tubes in the middle. The EDD disassembles into three individual pieces without causing bleeding in the patient. One of holders remains attached to the patient. The patient can now leave the hemodialysis machine and evacuate the danger zone.

The detailed manual for assembling the EDD is as follows:

Three holders (11) are placed flush against one another. Insert a spring (18) into the cavity (22) on each holder. Insert a sliding choker (15) into the cavity (22) on each of the two side holders (11), with the flat side next to the spring (18). Insert a sliding cutter (16) into the cavity (22) on the middle holder (11), with the flat side next to the spring (18). The three springs (18) are compressed by the sliding blocks (15) (16) against the blind end of the cavities until the sliding blocks (15) (16) are flush with the groove wall and the springs are located in the cavity (24) of the sliding blocks. Hold the three sliding blocks in place. Align the notches (23) on the three sliding blocks with the cam shift holes (13). Insert the special-shaped cam shift (19) with trigger (20) into the cam shift holes (13) on the holders, passing through the notches (23) on the sliding blocks (15) (16), to lock the sliding cutter (16) and the two sliding chokers (15) in place. Insert restraint lids (14) from the groove (12) side. The fixed blocks (17) are placed at the open end of the cavity (22) of the holders (11) and locked in place. The restraint lids (14) can slide between fixed blocks and holds. The EDD is now ready to use.

The detailed manual for using the EDD is as follows:

Insert the blood tubes (21 ) into the groove (12) and close the restraint lids (14) before a patient starts his hemodialysis treatment. When an emergency occurs, the patient turns the trigger handle (20), which turns the cam shift (19), releasing the sliding blocks (15) (16). The springs (18) push the sliding cutter (16) and chokers (15) against their corresponding fixed blocks (17). The two sliding chokers (15) in the side holders (11) choke off both blood tubes (21) in the groove. The sliding cutter (16) in the middle holder (11) simultaneously moves toward its corresponding fixed block (17) and severs the blood tubes (21). The three holders (11) disassemble into three individual pieces.

The new disengagement method using the EDD has many advantages over the conventional disengagement method. The EDD significantly improves the speed, safety, and ease of disengagement.

1. The Emergency-Disengagement Device (EDD) for patients undergoing hemodialysis comprises:
a. at least two holders within said device to enclose hemodialysis blood tubes,
b. at least two sliding chokers and at least one sliding cutter within said holders to permanently choke off blood flow and to sever said blood tubes upon releasing of a trigger handle,
c. device that disassembles into individual holders after use,
whereby said device will allow patients to rapidly disengage from hemodialysis machines to which they are connected, without causing bleeding.
